(19) United States
(12) Patent Application Publication
(10) Pub. No.: US 2024/0177857 A1
(43) Pub. Date: May 30, 2024

(54) METHOD, APPARATUS AND PROGRAM FOR DETERMINING ORTHOSTATIC HYPOTENSION USING HEART RATE DATA

(71) Applicant: TOBEDTX, Seoul (KR)

(72) Inventors: Yang Koo KANG, Seoul (KR); Ki Doo NAM, Incheon (KR)

(73) Assignee: TOBEDTX, Seoul (KR)

(21) Appl. No.: 18/324,311

(22) Filed: May 26, 2023

(30) Foreign Application Priority Data
Nov. 25, 2022 (KR) 10-2022-0160732

(51) Int. Cl. G16H 50/20; A61B 5/00; A61B 5/024; A61B 5/11; G16H 50/70

(52) U.S. Cl. CPC G16H 50/20; A61B 5/024; A61B 5/112; A61B 5/7264; G16H 50/70

(57) ABSTRACT

The present invention relates to a method, an apparatus, and a program for determining orthostatic hypotension using heart rate data, which determines an orthostatic hypotension using heart rate data of a user measured by contact or non-contact. The method for determining orthostatic hypotension using heart rate data, according to one embodiment of the present invention, comprises the steps of: obtaining basic information of a user; collecting heart rate data of the user; obtaining activity information on an activity performed by the user during a process of collecting heart rate data; and determining orthostatic hypotension of the user by analyzing the basic information, the heart rate data, and the activity information.

### CROSS-REFERENCE TO RELATED APPLICATION

This application claims priority to and the benefit of Korean Patent Application No. 10-2022-0160732, filed on Nov. 25, 2022, the disclosure of which is incorporated herein by reference in its entirety.

### BACKGROUND

### 1. Field of the Invention

The present invention relates to a method, an apparatus, and a program for determining orthostatic hypotension using heart rate data, and more particularly, to a method, an apparatus, and a program for determining orthostatic hypotension using heart rate data, which determines an orthostatic hypotension using heart rate data of a user measured in contact or non-contact.

### 2. Discussion of Related Art

When the user is lying down, or when the user stands for a long time, the user's body is changed, for example, when the user suddenly stands while sitting, and the blood is naturally collected in the lower half of the body by gravity. Accordingly, the amount of blood entering the heart is reduced, but at this time, neural reflection in the human body operates normally and a constant blood pressure may be maintained. The low blood pressure generated when the blood pressure maintenance reflector has an obstacle is called orthostatic hypotension.

Orthostatic hypotension is defined as such that an increase in the heart rate for maintaining blood flow does not occur even though the systolic blood pressure drops by mmHg or more and the diastolic blood pressure drops by 10 mmHg or more due to standing up. The orthostatic hypotension may occur in various diseases related to the autonomic nervous system, such as Parkinson's disease, multiple atrophy, pure autonomic nerve failure, and diabetic autonomic neuropathy, and is associated with an increase in the risk of falls, cardiovascular events, and cognitive disorders, and thus it is necessary to detect and manage the orthostatic hypotension early.

In addition, the conventional method of screening for orthostatic hypotension was generally a method of performing an orthostatic test. Accordingly, a space in which the inspection may be performed is limited, and the inspection process takes a long time, and inconvenience of the inspector also has a big problem. Accordingly, methods for replacing the Head up tilt test have been devised, but even in this case, there is a disadvantage in that it is possible to determine whether or not the orthostatic hypotension occurs only when a separate event such as Valsalva maneuver is performed. In this case, in the case of an inspector who cannot normally perform the event, there is also a problem that the inspection is impossible. Accordingly, there is a need to develop a technology capable of solving these problems.

### SUMMARY OF THE INVENTION

The present invention has been made in an effort to provide a method, an apparatus, and a program for determining orthostatic hypotension using heart rate data, which determines an orthostatic hypotension using heart rate data of a user measured by contact or non-contact.

The problems to be solved of the present disclosure are not limited to the above-mentioned problems, and other problems not mentioned will be clearly understood by the skilled person in the art from the following description.

The method for determining orthostatic hypotension using heart rate data according to an embodiment of the present invention includes the steps of: obtaining basic information of a user; collecting heart rate data of the user; obtaining activity information on an activity performed by the user in the process of collecting heart rate data; and determining orthostatic hypotension of the user by analyzing the basic information, the heart rate data, and the activity information.

In addition, the step of determining orthostatic hypotension may include the steps of: extracting input data from the basic information, heart rate data, and activity information; inputting the input data to a pre-learned artificial intelligence model; and obtaining an output of the artificial intelligence model and determining orthostatic hypotension of the user based on the output.

In addition, the method may further include a step of collecting data for determining orthostatic hypotension for a plurality of testers, wherein the step of collecting the data may include the steps of obtaining basic information of each testers, collecting heart rate data according to operations performed by the testers and operations performed by the testers, and determining orthostatic hypotension of each testers.

In addition, the step of obtaining basic information of each testers may include the steps of: receiving physical information and disease information of the testers; and classifying the testers according to a predetermined criterion based on the physical information and the disease information, and the step of determining orthostatic hypotension of the testers may include the steps of calculating a possibility of occurrence of the orthostatic hypotension for each group classified according to a predetermined criterion based on the heart rate data.

In addition, the step of determining orthostatic hypotension of the testers may include the steps of: extracting an operation in which the orthostatic hypotension occurs in each of the classification groups and a time during which the operation is performed; and calculating an average of heart rate data collected when the operation is performed in each of the classification groups, and the step of determining orthostatic hypotension of the user may include the steps of: determining a classification group in which the user is included; comparing heart rate data collected when the user performs an operation in which the orthostatic hypotension occurs in the classification group in which the user is included with the average of the heart rate data; and determining orthostatic hypotension of the user according to a comparison result.

In addition, the step of determining orthostatic hypotension of the testers may include the steps of: determining an operation in which the orthostatic hypotension has been generated by the testers included in each of the classification groups; and setting a weight value with respect to the operation in which the orthostatic hypotension has been generated, and the step of determining the user's orthostatic hypotension may include: determining a classification group in which the user is classified based on basic information of the user; assigning a score to heart rate data collected when the user performs a plurality of operations according to a score and a weight value corresponding to the classification group in which the user is included; and determining that the user is the orthostatic hypotension when a score obtained by integrating scores of the plurality of operations exceeds a preset score.

In addition, the step of collecting heart rate data may include a step of collecting data obtained by sensing the heart rate data of the tester from a sensor attached to the body of the tester or an apparatus for measuring the heart rate data of the tester in a non-contact manner at a position spaced apart from the tester, wherein the step of determining orthostatic hypotension of the testers may include the steps of: providing questions about an operation, a respiration method, and whether symptoms have occurred to each testers; obtaining an answer to the questions from each testers; and determining whether the orthostatic hypotension has occurred in each testers on the basis of the answer and extracting the heart rate data when the orthostatic hypotension has occurred.

In addition, the step of extracting the heart rate data when the orthostatic hypotension occurs may include the steps of: determining a heart rate data pattern when the orthostatic hypotension occurs and before and after the occurrence of the orthostatic hypotension; dividing the heart rate data pattern into a plurality of sections according to the heart rate data pattern; and setting a range for determining orthostatic hypotension in each of the plurality of sections and a heart rate data difference range between the plurality of sections for determining orthostatic hypotension on the basis of basic information of the testers.

In addition, the step of determining orthostatic hypotension of the user may include the steps of: determining a heart rate data area having a pattern similar to the heart rate data pattern from the collected heart rate data of the user; dividing the heart rate data area into a plurality of sections according to the pattern of the heart rate data area; extracting a difference between the heart rate data in each of the plurality of sections and the heart rate data between the plurality of sections; and determining orthostatic hypotension of the user by determining whether the extracted data value is within the predetermined range.

In addition, the step of determining orthostatic hypotension may include the steps of: extracting at least one tester having basic information similar to the basic information of the user; collecting heart rate data of the user when the user performs a plurality of operations; selecting one tester having heart rate data similar to the heart rate data of the user for the plurality of operations among the extracted testers; and determining orthostatic hypotension of the user according to a determination of the orthostatic hypotension of the selected tester.

An apparatus for determining orthostatic hypotension using heart rate data according to an embodiment of the present invention includes: a memory configured to store one or more instructions; and a processor configured to execute the one or more instructions stored in the memory, wherein the processor executes the one or more instructions to perform a method for determining orthostatic hypotension using heart rate data.

A program for determining orthostatic hypotension using heart rate data according to an embodiment of the present invention for solving the above-described problems may be combined with a computer that is a hardware, and may be stored in a recording medium readable by the computer so as to perform a method of determining orthostatic hypotension using heart rate data.

Other detailed matters of the present invention are included in the detailed description and the drawings.

### BRIEF DESCRIPTION OF THE DRAWINGS

FIG. 1 is a diagram illustrating a system for determining orthostatic hypotension using heart rate data according to an embodiment of the present invention.

FIG. 2 is a hardware configuration diagram of a determination apparatus according to an embodiment of the present invention.

FIG. 3 is a diagram illustrating a method for determining orthostatic hypotension using heart rate data according to an embodiment of the present invention.

FIG. 4 is a diagram illustrating a method of determining orthostatic hypotension of a user according to another embodiment of the present invention.

FIG. 5 is a diagram illustrating a method of determining orthostatic hypotension of a user according to another embodiment of the present invention.

FIG. 6 is a diagram illustrating a method of determining orthostatic hypotension of a user according to another embodiment of the present invention.

FIG. 7 is a diagram illustrating a method of determining orthostatic hypotension of a user according to another embodiment of the present invention.

### DETAILED DESCRIPTION OF EXEMPLARY EMBODIMENTS

Advantages and features of the present invention and methods of achieving the same will become apparent with reference to embodiments described in detail below together with the accompanying drawings. However, the present invention is not limited to the embodiments disclosed below, but may be implemented in various different forms, and the present embodiments are provided so that the disclosure of the present invention is complete and the scope of the present invention is completely known to a skilled person in the art in the technical field to which the present invention belongs, and the present invention is defined only by the scope of Claims.

The terminology used herein is for the purpose of describing embodiments and is not intended to be limiting of the present invention. In the specification, a singular form includes a plural form unless specifically mentioned in the text. The terms “comprises” and/or “comprising” used in the specification do not exclude the presence or addition of one or more other constituent elements in addition to the mentioned constituent elements. Throughout the specification, like reference numerals refer to like feature elements, and “and/or” includes each and every combination of the stated elements. Although “first”, “second”, and the like are used to describe various components, these components are not limited by these terms. These terms are only used to distinguish one component from another. Therefore, it is obvious that the first component mentioned below may be the second component within the technical spirit of the present invention.

Unless otherwise defined, all terms (including technical and scientific terms) used herein have the same meaning as commonly understood by skilled people in the art in the technical field to which the present invention belongs. In addition, terms defined in commonly used dictionaries are not ideally or excessively interpreted unless they are clearly specifically defined.

As used herein, the term “unit” or “module” refers to software, a hardware component such as FPGA or ASIC, and the term “unit” or “module” performs certain roles. However, the term “unit” or “module” is not limited to software or hardware. The “unit” or “module” may be configured to be in an addressable storage medium, or may be configured to reproduce one or more processors. Thus, for example, the “unit” or “module” includes components such as software components, object-oriented software components, class components, and task components, processes, functions, attributes, procedures, subroutines, segments of program code, drivers, firmware, microcode, circuits, data, databases, data structures, tables, arrays, and variables. The functionality provided within the components and “units” or “modules” may be combined into a smaller number of components and “units” or “modules” or further separated into additional components and “units” or “modules”.

In the present specification, a computer means all kinds of hardware devices including at least one processor, and may be understood as encompassing a software configuration operating in a corresponding hardware device according to an embodiment. For example, a computer may be understood as including all of a smartphone, a tablet PC, a desktop, a laptop, and a user client and an application driven in each device, but is not limited thereto.

Hereinafter, embodiments of the present disclosure will be described in detail with reference to the accompanying drawings.

Although each step described in the specification is described as being performed by a computer, the subject of each step is not limited thereto, and at least a part of each step may be performed in different devices according to an embodiment.

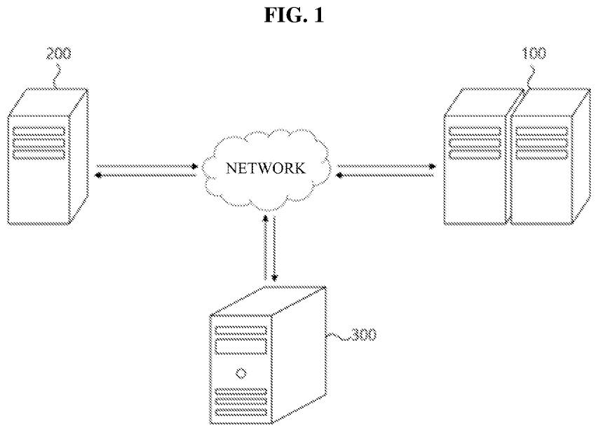

FIG. 1 is a diagram illustrating a system for determining orthostatic hypotension using heart rate data according to an embodiment of the present invention.

Referring to FIG. 1, the system for determining orthostatic hypotension using heart rate data according to an embodiment of the present invention may include a determination device 100, a user terminal 200, and an external server 300.

Here, the system for determining orthostatic hypotension using heart rate data shown in FIG. 1 is according to an embodiment, and the feature element thereof is not limited to the embodiment shown in FIG. 1, and may be added, changed, or deleted, as necessary.

The determination device 100 may obtain basic information of the user, collect heart rate data of the user, obtain activity information on an activity performed by the user in the process of collecting heart rate data, and analyze the basic information, the heart rate data, and the activity information to determine orthostatic hypotension of the user.

The user terminal 200 may access a website through a network, and may receive an orthostatic hypotension determination service using heart rate data from the determination device 100.

The user terminal 200 may include at least one of a smartphone including a display, a tablet PC, a desktop computer, and a laptop computer in at least a part of the user terminal 200, and may be provided with an orthostatic hypotension determination service using heart rate data provided from the determination device 100 in a process of executing a browser. However, it is not limited thereto.

The external server 300 may be connected to the determination device 100 through a network, and may store and manage various pieces of information for the determination device 100 to perform the method of determining orthostatic hypotension using heart rate data.

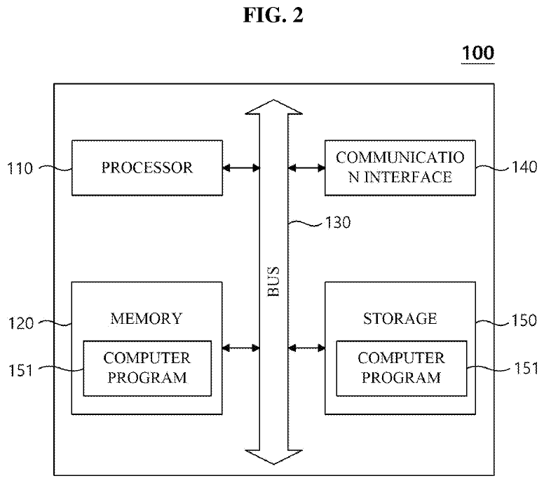

In addition, the external server 300 may receive and store various information and data generated as the determination device 100 performs the method of determining orthostatic hypotension using heart rate data. For example, the external server 300 may be a storage server separately provided outside the determining device 100. Referring to FIG. 2, hardware feature of the determining device 100 will be described.

FIG. 2 is a hardware configuration diagram of a determination apparatus according to an embodiment of the present invention.

Referring to FIG. 2, the determination device 100 (hereinafter, referred to as a computing device) according to an embodiment of the present disclosure may include one or more processors 110, a memory 120 for loading a computer program 151 executed by the processors 110, a bus 130, a communication interface 140, and a storage 150 for storing the computer program 151. Here, only components related to the embodiment of the present invention are shown in FIG. 2. Therefore, it may be understood by those skilled in the art that other general-purpose feature elements may be further included in addition to the elements illustrated in FIG. 2.

The processor 110 controls the overall operation of each feature of the computing device 100. The processor 110 may include a central processing unit (CPU), a micro processor unit (MPU), a micro controller unit (MCU), a graphic processing unit (GPU), or any type of processor well known in the technical field of the present invention.

In addition, the processor 110 may perform an operation on at least one application or program for executing a method according to embodiments of the present disclosure, and the computing device 100 may include one or more processors.

In various embodiments, the processor 110 may further include a Random Access Memory (RAM, not shown) and a Read-Only Memory (ROM, not shown) for temporarily and/or permanently storing signals (or data) processed in the processor 110. In addition, the processor 110 may be implemented in the form of a system on chip (SoC) including at least one of a graphic processor, a RAM, and a ROM.

The memory 120 stores various data, commands, and/or information. The memory 120 may load the computer program 151 from the storage 150 to execute the method/operation according to various embodiments of the present disclosure. When the computer program 151 is loaded into the memory 120, the processor 110 may perform the method/operation by executing one or more instructions constituting the computer program 151. The memory 120 may be implemented as a volatile memory such as a RAM, but the technical scope of the present disclosure is not limited thereto.

The bus 130 provides a communication function between feature elements of the computing device 100. The bus 130 may be implemented as various types of buses such as an address bus, a data bus, and a control bus.

The communication interface 140 supports wired/wireless Internet communication of the computing device 100. In addition, the communication interface 140 may support various communication methods other than Internet communication. To this end, the communication interface 140 may include a communication module well known in the technical field of the present invention. In some embodiments, the communication interface 140 may be omitted.

The storage 150 may non-temporarily store the computer program 151. When the method of determining orthostatic hypotension using the heart rate data is performed through the computing apparatus 100, the storage 150 may store various information necessary for providing the method of determining orthostatic hypotension using the heart rate data.

The storage 150 may be configured to include a non-volatile memory such as a read only memory (ROM), an erasable programmable ROM (EPROM), an electrically erasable programmable ROM (EEPROM), a flash memory, or the like, a hard disk, a removable disk, or any form of computer-readable recording medium well known in the technical field to which the present invention pertains.

The computer program 151 may include one or more instructions that, when loaded into the memory 120, cause the processor 110 to perform a method/operation according to various embodiments of the present disclosure. That is, the processor 110 may perform the method/operation according to various embodiments of the disclosure by executing the one or more instructions.

According to an embodiment, the computer program 151 may include one or more instructions for performing the method of determining orthostatic hypotension using the heart rate data, the method including the steps of: obtaining basic information of the user; collecting heart rate data of the user; obtaining information on an activity performed by the user in a process of collecting heart rate data; and determining orthostatic hypotension of the user by analyzing the basic information, the heart rate data, and the activity information.

The steps of the method or algorithm described in relation to the embodiment of the present disclosure may be implemented directly in hardware, implemented in a software module executed by hardware, or implemented in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any form of computer-readable recording medium well known in the art to which the present invention pertains.

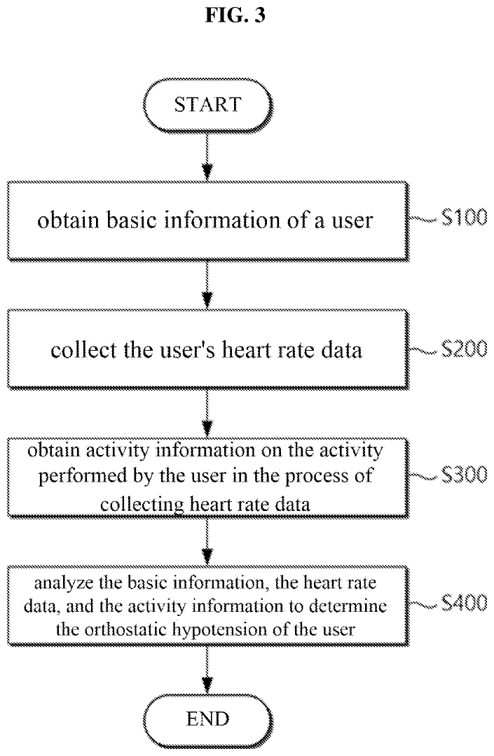

The feature elements of the present invention may be implemented as a program (or an application) to be executed in combination with a computer that is hardware, and may be stored in a medium. The feature elements of the present invention may be implemented using software programming or software elements, and similarly, the embodiments may be implemented with any programming or scripting language such as C, C++, Java, assembler language, or the like, with the various algorithms being implemented with any combination of data structures, objects, processes, routines, or other programming elements. Functional aspects may be implemented with algorithms executed on one or more processors. Hereinafter, a method of determining orthostatic hypotension using heart rate data provided by the computing apparatus 100 will be described with reference to FIG. 3.

FIG. 3 is a diagram illustrating a method for determining orthostatic hypotension using heart rate data according to an embodiment of the present invention.

Referring to FIG. 3, the computing device 100 may obtain basic information of a user (S100). The basic information of the user may include physical information including gender, age, height, weight, and blood pressure, and disease information including disease information of the user. The computing device 100 may provide an instruction for inputting basic information to the user terminal 200 of the user, and may obtain the basic information of the user input through the user terminal 200. Meanwhile, the computing device 100 may provide an instruction for inputting basic information to the user terminal 200 of the manager who manages a process of determining orthostatic hypotension of the user, and obtain the basic information of the user that the manager inputs through the user terminal 200. Meanwhile, the administrator may be an expert such as a doctor or the like capable of determining orthostatic hypotension according to the state of the user, but is not limited thereto.

The computing device 100 may collect the user's heart rate data (S200). The computing device 100 may collect data obtained by sensing the heart rate data of the user from a sensor attached to the body of the user or a device for measuring the heart rate data of the tester in a non-contact manner at a position spaced apart from the user. For example, the user may wear a wearable device on a hand, a head, or the like, or may attach a sensor capable of measuring separate heart rate data to the body of the user, and the sensor that contacts the body of the user may periodically measure the heart rate data of the user and transmit the measured heart rate data to the computing apparatus 100.

As another example, the method may further include analyzing heart rate data of the user from an image obtained by photographing the user by a camera installed at a position spaced apart from the user, analyzing heart rate data of the user from a voice of the user obtained by a microphone installed at a position spaced apart from the user, and analyzing the heart rate data of the user from an ultrasonic wave emitted from an ultrasonic device installed at a position spaced apart from the user and then returned to the user. Analyzing the heart rate data through the camera, the microphone, and the ultrasound device may be performed using at least one device. Meanwhile, each of the camera, the microphone, and the ultrasound device may analyze the user's heart rate data by using the obtained data, transmit the obtained data to a separate external server, and analyze the user's heart rate data by using the data obtained from the external server. Here, the heart rate data may include a heart rate, a heartbeat time, a heartbeat interval, and the like, but is not limited thereto.

The computing device 100 may obtain activity information on the activity performed by the user in the process of collecting heart rate data (S300). The computing device 100 may obtain activity information from the user when an issue occurs in the heart rate data after the collection of the heart rate data for the user is completed and a specific activity is terminated. The computing device 100 may provide an instruction for inputting activity information on the activity performed by the user terminal 200, and may obtain the activity information through the user terminal 200. The activity information may include sleeping, having meals, walking, standing up, and the like, and may include a respiration state such as comfortable respiration, gasping respiration, and Valsalva maneuver. In addition, the computing apparatus 100 may obtain activity information of the user by using a sensor of the wearable device.

In addition, the computing device 100 may obtain the activity information of the user from a camera that photographs the user or a sensor that senses the motion of the user, and may obtain the activity information of the user at the time point at which the heart rate data is measured by using data obtained by photographing or sensing the motion of the user corresponding to the time point at which the heart rate data of the user is measured.

The feature of the present invention enables a user to determine orthostatic hypotension through natural daily life without a separate event such as Head up tilt test or Valsalva maneuver, thereby increasing user convenience, and examining the symptoms generated in the daily life of a user.

Therefore, the above-described steps of S200 to S300 may be performed in a process in which the user performs daily life.

For example, after allowing the user to perform a natural daily life in an environment (e.g., an environment in which the user wears a wearable device or in which a non-contact heart rate data measuring device is installed) in which heart rate data may be measured in a contact or non-contact manner, information about what activity the user performed during a corresponding period may be obtained through a questionnaire at a specific time point. Alternatively, the behavior information of the user corresponding to the heart rate data may be obtained using a wearable device worn by the user.

The computing device 100 may obtain the collected heart rate data and information on the behavior of the user corresponding to each time point. Through this, the computing device 100 may obtain heart rate data information according to the behavior obtained while the user performs daily life, process the heart rate data information, and obtain input data that can be input to the artificial intelligence model or obtain basic data for statistical analysis.

The computing device 100 may input the obtained data to the artificial intelligence model to obtain an output, or may determine the orthostatic hypotension of the user through a preset statistical analysis method or comparison with an existing database.

In addition, the computing device 100 may guide an activity to be performed by the user, collect heart rate data of the user while the user performs the corresponding activity, and obtain activity information performed by the user in the process of collecting heart rate data.

The computing device 100 may analyze the basic information, the heart rate data, and the activity information to determine the orthostatic hypotension of the user (S400).

The computing device 100 may extract input data from the basic information, the heart rate data, and the activity information, and may input the input data to the pre-learned artificial intelligence model. The computing device 100 may convert the basic information, the heart rate data, and the activity information into the form of input data in order to input the same into a pre-learned artificial intelligence model, and may input the converted input data into the artificial intelligence model.

The computing device 100 may obtain an output of the artificial intelligence model and determine the orthostatic hypotension of the user based on the output. The artificial intelligence model may output an output value for input data according to a learned result, and the computing device 100 may determine the orthostatic hypotension of the user based on the output value.

The artificial intelligence model may be a model generated by learning a result of determining orthostatic hypotension for a plurality of testers. In detail, the computing device 100 may obtain basic information of each of a plurality of testers. The basic information of each of the plurality of testers may include physical information including gender, age, height, weight, and blood pressure, and disease information including disease information of the plurality of testers. A method of obtaining basic information from a plurality of testers may be performed in the same manner as the method described in step S100.

In various embodiments, the artificial intelligence model may include at least one neural network.

Throughout the specification, a computation model, a neural network, a network function, and a neural network may be used as the same meaning. A neural network may be configured as a set of interconnected computational units that may generally be referred to as “nodes”. These “nodes” may be referred to as “neurons”. The neural network includes at least one node. Nodes (or neurons) constituting neural networks may be interconnected by one or more “links”.

A deep neural network (DNN) may refer to a neural network including a plurality of hidden layers in addition to an input layer and an output layer. Using deep neural networks, potential structures of data can be identified. That is, the potential structure of a picture, text, video, voice, and music (for example, which object is in the picture, what is the content and emotion of the text, what is the content and emotion of the voice, etc.) may be identified. The deep neural network may include a convolutional neural network (CNN), a recurrent neural network (RNN), an auto encoder, Generative Adversarial Networks (GAN), a restricted boltzmann machine (RBM), a deep belief network (DBN), a Q network, a U network, a Siamese network, and the like. The description of the deep neural network described above is only an example, and the present invention is not limited thereto.

The neural network may be learned by at least one of supervised learning, unsupervised learning, and semi-supervised learning. The learning of the neural network is to minimize an error in output. In the learning of the neural network, the learning data is repeatedly inputted to the neural network, errors of the output and target of the neural network with respect to the learning data are calculated, and the errors of the neural network are backpropagated from the output layer of the neural network toward the input layer in a direction to reduce the errors, thereby updating the weight of each node of the neural network. In the case of teacher learning, learning data in which the correct answer is labeled is used (that is, labeled learning data) for each learning data, and in the case of unsupervised learning, the correct answer may not be labeled on each learning data. That is, for example, in the case of teacher learning about data classification, the learning data may be data in which a category is labeled on each of the learning data. The labeled learning data may be input to the neural network, and an error may be calculated by comparing an output (category) of the neural network with a label of the learning data. As another example, in the case of unsupervised learning about data classification, an error may be calculated by comparing the learning data, which is an input, with the neural network output. The calculated error is reversely propagated in the neural network (i.e., in the direction from the output layer to the input layer), and the connection weight of each node of each layer of the neural network may be updated according to the reverse propagation. A change amount of the connection weight of each node to be updated may be determined according to a learning rate. The calculation of the neural network for the input data and the backpropagation of the error may constitute a learning cycle (epoch). The learning rate may be differently applied according to the number of repetitions of the learning cycle of the neural network. For example, in an early stage of learning of the neural network, a high learning rate may be used to allow the neural network to quickly secure a predetermined level of performance, thereby increasing efficiency, and in a late stage of learning, a low learning rate may be used to increase accuracy.

In learning of a neural network, generally, learning data may be a subset of actual data (that is, data to be processed using the learned neural network), and thus, there may be a learning cycle in which an error for the learning data is reduced but an error for the actual data is increased. Overfitting is a phenomenon in which errors in actual data increase by learning excessively on learning data. For example, a phenomenon in which a neural network that learns a cat by seeing a yellow cat does not recognize that the neural network is the cat when seeing a cat other than the yellow cat may be a kind of overfitting. Overfitting can act as a cause of increasing machine learning algorithm errors. In order to prevent the overfitting, various optimization methods may be used. In order to prevent overfitting, a method of increasing learning data, regularization, and dropout in which a part of a node of a network is omitted in a learning process may be applied.

Throughout the specification, a computation model, a neural network, a network function, and a neural network may be used as the same meaning. A data structure may include a neural network. The data structure including the neural network may be stored in a computer-readable medium. The data structure including the neural network may also include data input to the neural network, a weight of the neural network, a hyper parameter of the neural network, data obtained from the neural network, an activity function associated with each node or layer of the neural network, a loss function for learning of the neural network. A data structure including a neural network may include any of the feature elements of the configurations disclosed above. That is, the data structure including the neural network may include all or any combination of data input to the neural network, a weight of the neural network, a hyper parameter of the neural network, data obtained from the neural network, an activity function associated with each node or layer of the neural network, a loss function for learning the neural network, and the like. In addition to the above-described configurations, the data structure including the neural network may include any other information that determines the characteristics of the neural network. In addition, the data structure may include all types of data used or generated in the computation process of the neural network, and is not limited to the above-described matters. The computer readable medium may include a computer readable recording medium and/or a computer readable transmission medium. A neural network may be configured as a set of interconnected computation units, which may generally be referred to as nodes. These nodes may be referred to as neurons. The neural network includes at least one node.

The data structure may include data input to a neural network. A data structure including data input to a neural network may be stored in a computer-readable medium. The data input to the neural network may include learning data input in a neural network learning process and/or input data input to a neural network in which learning has been completed. The data input to the neural network may include pre-processed data and/or data to be pre-processed. The pre-processing may include a data processing process for inputting data into the neural network. Accordingly, the data structure may include data to be pre-processed and data generated by pre-processing. The above-described data structure is only an example, and the present invention is not limited thereto.

The data structure may include a weight of the neural network. In this specification, a weight and a parameter may be used as the same meaning.) A data structure including a weight of a neural network may be stored in a computer-readable medium. The neural network may include a plurality of weights. The weight may be variable, and may be variable by a user or an algorithm in order for the neural network to perform a desired function. For example, when one or more input nodes are connected to one output node by each link, the output node may determine an output node value based on values input to input nodes connected to the output node and a parameter set to a link corresponding to each input node. The above-described data structure is only an example, and the present invention is not limited thereto.

As an example, and not by way of limitation, the weight may include a weight that varies in a neural network learning process and/or a weight for which neural network learning has been completed. The weight varied in the neural network learning process may include a weight at a time point when a learning cycle starts and/or a weight varied during the learning cycle. The weight for which the neural network learning is completed may include a weight for which the learning cycle is completed. Accordingly, the data structure including the weight of the neural network may include a data structure including a weight that varies in a neural network learning process and/or a weight for which neural network learning has been completed. Therefore, it is assumed that the above-described weights and/or the combination of the weights are included in the data structure including the weights of the neural network. The above-described data structure is only an example, and the present invention is not limited thereto.

The data structure including the weight of the neural network may be stored in a computer-readable storage medium (e.g., a memory or a hard disk) after a serialization process. The serialization may be a process of storing a data structure in the same or different computing devices and converting the data structure into a form that can be reconfigured and used later. The computing device may serialize the data structure to transmit and receive data through a network. The data structure including the weights of the serialized neural network may be reconfigured in the same computing device or different computing devices through deserialization. The data structure including the weight of the neural network is not limited to serialization. Further, the data structure including the weight of the neural network may include a data structure (e.g., B-Tree, Trie, m-way search tree, AVL tree, and Red-Black Tree in a non-linear data structure) for increasing the efficiency of calculation while minimally using the resources of the computing device. The foregoing description is merely an example, and the present invention is not limited thereto.

The data structure may include a hyper-parameter of the neural network. The data structure including the hyper parameter of the neural network may be stored in a computer-readable medium. The hyper parameter may be a variable varied by a user. The hyper parameter may include, for example, a learning rate, a cost function, the number of learning cycle iterations, weight initialization (e.g., setting a range of a weight value to be weight initialization), and the number of Hidden Unit (e.g., the number of hidden layers and the number of nodes of a hidden layer). The above-described data structure is only an example, and the present invention is not limited thereto.

The computing device 100 may extract input data of the artificial intelligence model from the basic information, the heart rate data, and the activity information. The input data may be configured in the form of a vector including values extracted based on at least some of the basic information, heart rate data, and activity information, but is not limited thereto.

In various embodiments, the computing device 100 may extract, from the heart rate data, an average in the time domain, a maximum, a minimum, a standard deviation of all NN intervals (SDNN), a standard deviation of differences between adjacent NN intervals (SDSD), the square root of the mean of the sum of the squares of differences between adjacent NN intervals (RMSSD), a count divided by the total number of all NN intervals (PNN50), a count Number of pairs of adjacent NN intervals differing by more than 50 ms in the entire recording (NN50), a count divided by the total number of all NN intervals (PNN20), a count Number of pairs of adjacent NN intervals differing by more than 20 ms in the entire recording (NN20), or a percentile value according to a specific criterion.

In addition, the computing device 100 may extract total power, very low frequency bands power (VLF), low frequency bands power (LF), high frequency bands power (HF), LF/HF, normalized If power (NLF), normalized hf power (NHF) values, and the like in the frequency domain from the heart rate data, and may also extract various values including C. HRV triangular index, sampen, Cardiac Pathmatic Index (CSI), Cadiac Vagal Index (CVI), and Modified CSI, but is not limited thereto.

In addition, the user's basic information and activity information may be converted into a specific value according to a preset criterion.

The computing device 100 may learn the artificial intelligence model by using input data which includes the extracted values and learning data in which the orthostatic hypotension corresponding to the input data is labeled.

The learned artificial intelligence model may output whether an orthostatic hypotension corresponding to input data extracted from user information is low blood pressure.

In various embodiments, the computing device 100 may generate a plurality of input data for each operation of the user. For example, the user may stand during the measurement period, lie down, sit, stand up, and breathe in a Valsalva maneuver. In this case, the computing device 100 may generate input data according to the heart rate data for each operation time point of the user.

In addition, the computing device 100 may define an additional operation corresponding to the change in the operation when the operation of the user is changed. For example, when the user stands up while sitting, an additional operation of “standing up while sitting down” may be defined, and another input data based on heart rate data for a preset time after the corresponding operation change may be generated.

The computing device 100 may obtain output data obtained through the artificial intelligence model from each input data. The computing device 100 may assign a weight according to an operation of input data corresponding to each output data to each output data. For example, a high weight may be given to an operation in which the orthostatic hypotension is well detected. The computing device 100 may determine whether the user has the orthotropic hypotension based on the weighted output data (e.g., deriving an average value, counting the number of output data exceeding a preset reference value, comparing the number of output data with a reference value of the orthotropic hypotension determination, etc.).

In various embodiments, the computing device 100 may calculate a first orthostatic hypotension score of the user based on output data obtained from input data corresponding to the stationary operation, and may calculate a second orthostatic hypotension score of the user based on output data obtained from input data corresponding to the operation change time point. The computing device 100 may determine that the user is in the orthotropic hypotension when both the first orthotropic hypotension score and the second orthotropic hypotension score are equal to or higher than a predetermined reference value, determine that the user is in the orthotropic hypotension risk group when the second orthotropic hypotension score is equal to or higher than the predetermined reference value and the first orthotropic hypotension score is lower than the predetermined reference value, and determine that the user is normal when both the two scores are lower than the predetermined reference value. In addition, when the first orthotropic hypotension score is higher than the second orthotropic hypotension score, re-measurement may be requested.

The computing device 100 may collect heart rate data according to operations performed by the testers and operations performed by the testers. A method of collecting heart rate data according to the operations performed by the plurality of testers and the operations performed by the testers may be performed in the same manner as the method described in steps S200 and S300.

In addition, the computing device 100 may provide questions about operations, respiration methods, and whether symptoms have occurred to each testers, and may obtain answers to the questions from each testers. For example, the computing device 100 may ask a question about whether a symptom related to the orthostatic hypotension occurs when the testers perform an operation or a respiration method, and may match an answer to the question with the operation or respiration method performed by the testers and store the answer.

The computing device 100 may determine the orthostatic hypotension of the testers. The computing device 100 may analyze the basic information, the heart rate data, and the activity information to determine the orthostatic hypotension of the testers, and may determine the orthostatic hypotension of each of the plurality of testers by an expert.

The computing device 100 may learn a result of determining orthostatic hypotension with respect to basic information, heart rate data, and activity information for each testers to an artificial intelligence model, and to this end, may convert each of the basic information, the heart rate data, and the activity information into a form of input data and use the form of the input data as an input value of the artificial intelligence model, and may convert the result of determining orthostatic hypotension into a form of output data and use the form of the output data as an output value of the artificial intelligence model.

By inputting the basic information, heart rate data, and activity information of the user as input data to the artificial intelligence model learned through such a process, a result of determining the orthostatic hypotension of the user may be output.

In addition, the computing device 100 may determine whether each testers has an orthostatic hypotension on the basis of an answer to the question provided to the testers, and extract heart rate data when the orthostatic hypotension is generated. The computing device 100 may learn the activity information and heart rate data of the testers when the orthostatic hypotension occurs to the artificial intelligence model. To this end, the computing device 100 may convert each of the basic information, heart rate data, and activity information of the testers into the form of input data to use it as an input value of the artificial intelligence model, and may convert the result of the determination of the orthostatic hypotension into the form of output data to use it as an output value of the artificial intelligence model.

By inputting the basic information, heart rate data, and activity information of the user as input data to the artificial intelligence model learned through such a process, a result of determining orthostatic hypotension of the user may be output.

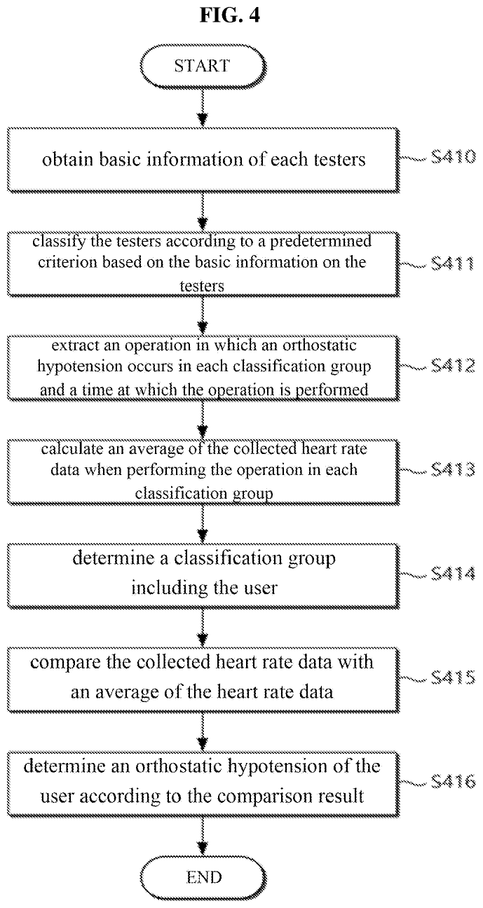

FIG. 4 is a diagram illustrating a method of determining orthostatic hypotension of a user according to another embodiment of the present invention.

Referring to FIG. 4, the computing device 100 may obtain basic information of each testers (S410). A method of obtaining basic information from a plurality of testers may be performed in the same manner as the method described in step S100.

The computing device 100 may classify the testers according to a predetermined criterion based on the basic information on the testers (S411). That is, the average heart rate of a woman and a man may be different, and the average heart rate may also be different according to a height, a weight, an age, and the like, and the average heart rate may also be different according to disease information of a tester.

Accordingly, the computing apparatus 100 may classify the testers according to a preset criterion and classify testers having similar heart rates. For example, it is possible to classify testers according to age, testers having a specific disease, or testers according to gender. Furthermore, it is possible to classify the testers according to various criteria. For example, testers who are male and in their 20s, and testers who are male and in their 30s may be classified.

The computing device 100 may calculate the possibility of occurrence of orthostatic hypotension for each classification group classified according to a predetermined criterion based on the heart rate data. The computing apparatus 100 may calculate the possibility of occurrence of the orthostatic hypotension for each classification group based on a result of determining orthostatic hypotension of the testers included in each classification group. This may be used as an index for helping the user determine the orthostatic hypotension and may also be used as an index for health management of the user.

The computing device 100 may extract an operation in which an orthostatic hypotension occurs in each classification group and a time at which the operation is performed (S412). The computing device 100 may determine an operation in which the orthostatic hypotension is generated with respect to the testers in which the orthostatic hypotension is generated among testers included in each classification group. For example, if orthostatic hypotension occurred when one tester included in a specific classification group stood up quickly while sitting down and when another tester sat up while lying down, the computing device 100 may determine that standing up quickly while sitting down and sitting down while lying down are the operations causing orthostatic hypotension in the specific classification group. In addition, the computing device 100 may extract the time when the corresponding operations are performed to clarify the operation in which the orthostatic hypotension is generated. For example, when the patient slowly stands up while sitting, the probability of generating an orthostatic hypotension is low, whereas when the patient quickly stands upwhile sitting, the probability of generating an orthostatic hypotension may be high. Accordingly, by extracting the time during which the operation is performed, the operation in which the orthostatic hypotension is generated may be clearly defined.

The computing device 100 may calculate an average of the collected heart rate data when performing the operation in each classification group (S413). The computing device 100 may calculate an average of the heart rate data of the tester in which the orthostatic hypotension is generated.

The computing device 100 may determine a classification group including the user (S414). The computing device 100 may determine a classification group including the user based on the basic information obtained from the user.

The computing device 100 may compare heart rate data collected when the user performs an operation in which the orthostatic hypotension is generated in the classification group in which the user is included with an average of the heart rate data (S415). That is, the user may perform a plurality of operations in order to determine the orthostatic hypotension, and may collect heart rate data when the user performs an operation in which the orthostatic hypotension is generated in the classification group including the user among the plurality of operations performed by the user. For example, in the classification group including the user, when the operation in which the orthostatic hypotension occurs is an operation that occurs quickly after sitting down or an operation that occurs after lying down, heart rate data when the user performs the motion that occurs quickly after sitting down or the operation that occurs after lying down may be collected.

The computing device 100 may determine an orthostatic hypotension of the user according to the comparison result (S416). The computing device 100 may determine that the user's heart rate is the orthostatic hypotension when the user's heart rate data is less than the average of the heart rate data as a result of comparing the heart rate data collected when the user performs an operation in which the orthostatic hypotension is generated in the classification group including the user. In other words, in the case of the orthostatic hypotension, the blood collected under the body is not rapidly supplied to the brain when the user suddenly stands up, and thus the heart rate for supplying the blood can be increased. However, when the heart rate is not sufficiently fast, the blood is not supplied to the brain, and thus the orthostatic hypotension may be generated. Meanwhile, the computing device 100 may set a predetermined range based on the average of the heart rate data, and may determine whether the user is orthostatic hypotension, falls under orthostatic hypotension risk group, or is not orthostatic hypotension according to the range in which the user's heart rate data is included. However, the criteria for determining according to the range are not limited thereto.

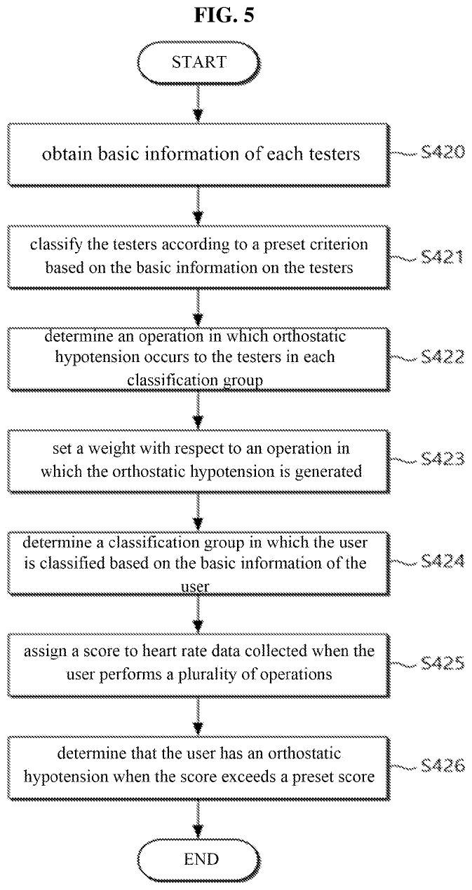

FIG. 5 is a diagram illustrating a method of determining orthostatic hypotension of a user according to another embodiment of the present invention.

Referring to FIG. 5, the computing device 100 may obtain basic information of each testers (S420). A method of obtaining basic information from a plurality of testers may be performed in the same manner as the method described in step S410.

The computing device 100 may classify the testers according to a preset criterion based on the basic information on the testers (S421). The method of classifying a plurality of testers according to a preset criterion may be performed in the same manner as the method described in step S411.

The computing device 100 may determine an operation in which orthostatic hypotension occurs to the testers in each classification group (S422). The computing device 100 may determine an operation in which the orthostatic hypotension is generated with respect to the testers in which the orthostatic hypotension is generated among testers included in each classification group. For example, if orthostatic hypotension occurred when one tester included in a specific classification group stood up quickly while sitting down and when another tester sat up while lying down, the computing device 100 may determine that standing up quickly while sitting down and sitting down while lying down are the operations causing orthostatic hypotension in the specific classification group.

The computing device 100 may set a weight with respect to an operation in which the orthostatic hypotension is generated (S423). The computing device 100 may assign a score to each operation performed by the testers in order to determine the orthostatic hypotension for each classification group, and may set a higher weight to an operation in which the orthostatic hypotension occurs among the plurality of operations. Meanwhile, in the case where many testers have an orthostatic hypotension for a specific operation, a higher weight may be set for the specific operation, but the present disclosure is not limited thereto.

Meanwhile, the score for each operation may be set to be given a higher score when the score is higher or lower based on the heart rate data of the testers in each operation, and may be given a higher score for a range in which the possibility of orthostatic hypotension is higher.

The computing device 100 may determine a score for each testers with respect to an operation performed by each testers, and may set a score for determining orthostatic hypotension low blood pressure based on the determined score. For example, since the scores of the test subjects having the non-occurrence of orthostatic hypotension have higher scores compared to those having the occurence of orthostatic hypotension, score for determining orthostatic hypotension can be set based on the score of the test subjects having the occurence of orthostatic hypotension. That is, the average of the scores of the testers in which the orthostatic hypotension occurs may be calculated as a score for determining orthostatic hypotension, or the lowest score among the testers in which the orthostatic hypotension occurs may be set as a score for determining orthostatic hypotension. However, it is not limited thereto.

The computing device 100 may determine a classification group in which the user is classified based on the basic information of the user (S424). The computing device 100 may determine a classification group including the user based on the basic information obtained from the user.

The computing device 100 may assign a score to heart rate data collected when the user performs a plurality of operations according to a score and a weight corresponding to a classification group in which the user is included (S425). The computing device 100 may assign a score to heart rate data collected when the user performs a plurality of operations according to a score and a weight assigned to each of the operations performed by the testers in order to determine the orthostatic hypotension in step S423. In this case, the score and the weight assigned to each operation may follow the score and the weight corresponding to the classification group including the user.

The computing device 100 may determine that the user has an orthostatic hypotension when the score obtained by integrating the scores for each of the plurality of operations exceeds a preset score (S426). The computing device 100 may determine whether the user has an orthostatic hypotension based on the score for determining orthostatic hypotension set in step S423. That is, when the integrated score according to the user's heart rate data exceeds the score for determining the preset orthostatic hypotension, it may be determined that the user is orthostatic hypotension.

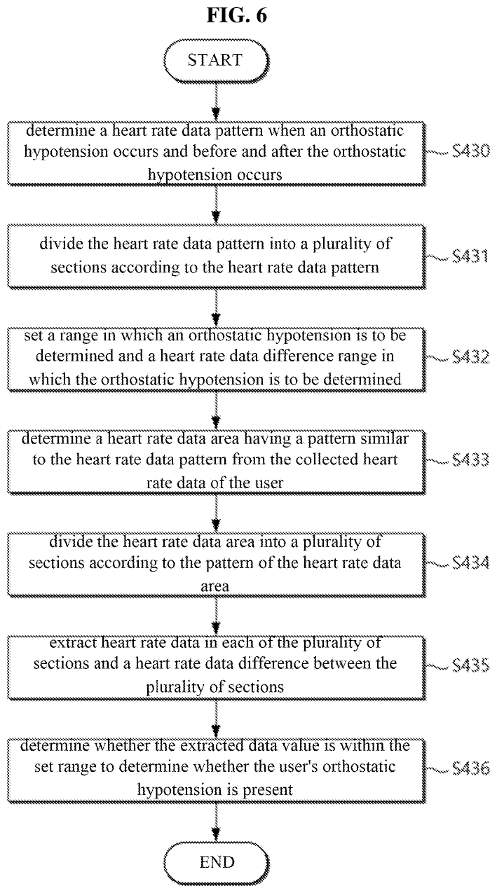

FIG. 6 is a diagram illustrating a method of determining orthostatic hypotension of a user according to another embodiment of the present invention.

Referring to FIG. 6, the computing device 100 may determine a heart rate data pattern when an orthostatic hypotension occurs and before and after the orthostatic hypotension occurs (S430). The computing device 100 may determine an orthostatic hypotension of the testers based on the basic information, the heart rate data, and the activity information, and analyze a heart rate data pattern before and after the orthostatic hypotension occurs when the orthostatic hypotension occurs. For example, when a tester who has been sitting down and performing a fast-acting operation generates orthostatic hypotension, the heart rate may be maintained constant and then rapidly increased.

The computing device 100 may divide the heart rate data pattern into a plurality of sections according to the heart rate data pattern (S431). For example, when the heart rate is rapidly increased after being constantly maintained, the section in which the heart rate is constantly maintained may be divided into one section, and the section in which the heart rate is rapidly increased may be divided into another section, but is not limited thereto.

The computing device 100 may set a range in which an orthostatic hypotension is to be determined in each of the plurality of sections and a heart rate data difference range between the plurality of sections in which the orthostatic hypotension is to be determined based on the basic information of the testers (S432).

The computing device 100 may analyze heart rate data in each section. For example, the minimum heart rate data, the maximum heart rate data, the average heart rate data, the difference between the maximum heart rate data and the minimum heart rate data, the pulse, and the like may be analyzed, and the maximum heart rate data difference between the sections, the minimum heart rate data difference between the sections, the average heart rate data difference between the sections, and the like may be analyzed, but the analyzed data may not be limited thereto.

The computing device 100 may set a range in which the orthostatic hypotension is to be determined in each of the plurality of sections and a heart rate data difference range between the plurality of sections in which the orthostatic hypotension is to be determined, based on the analyzed data. For example, in the case of the orthostatic hypotension, since the heart rate is not sufficient, blood is not supplied to the brain, and in the case of the user who generates the orthostatic hypotension, the heart rate may not be higher than that of the user who does not generate the orthostatic hypotension. Accordingly, a difference in heart rate between before and after the occurrence of the orthostatic hypotension may be less in the case of the user in which the orthostatic hypotension is generated than in the case of the user in which the orthostatic hypotension is not generated. The computing device 100 may set a difference range of heart rate data between a plurality of sections for which the orthostatic hypotension is to be determined using this point. However, it is not limited thereto.

The computing device 100 may determine a heart rate data area having a pattern similar to the heart rate data pattern from the collected heart rate data of the user (S433). For example, the computing device 100 may determine whether there is a heart rate data pattern in which the heart rate of the user is constantly maintained and then rapidly increased among the heart rate data patterns of the user.

The computing device 100 may divide the heart rate data area into a plurality of sections according to the pattern of the heart rate data area (S434). When there is a heart rate data area having a pattern similar to the heart rate data pattern in the heart rate data of the user, the computing device 100 may divide the corresponding heart rate data area into a plurality of sections. For example, when the heart rate is rapidly increased after being constantly maintained, the section in which the heart rate is constantly maintained may be divided into one section, and the section in which the heart rate is rapidly increased may be divided into another section, but is not limited thereto.

The computing device 100 may extract heart rate data in each of the plurality of sections and a heart rate data difference between the plurality of sections (S435). The computing device 100 may analyze heart rate data in each section. For example, the minimum heart rate data, the maximum heart rate data, the average heart rate data, the difference between the maximum heart rate data and the minimum heart rate data, the pulse, and the like may be analyzed, and the maximum heart rate data difference between the sections, the minimum heart rate data difference between the sections, the average heart rate data difference between the sections, and the like may be analyzed, but the analyzed data may not be limited thereto.

The computing device 100 may determine whether the extracted data value is within the set range to determine whether the user's orthostatic hypotension is present (S436). The computing device 100 may determine whether the analyzed data is within a range in which the orthostatic hypotension is to be determined and a heart rate data difference range between the plurality of sections in which the orthostatic hypotension is to be determined in each of the plurality of sections set in step S432, and determine that the user has the orthostatic hypotension when the analyzed data is within the set range. Meanwhile, a plurality of data to be compared may be determined as the orthostatic hypotension when the plurality of data satisfy all of the conditions, but may be determined as the orthostatic hypotension when the predetermined number of pieces of data satisfy the conditions. In addition, a weight may be assigned to each condition, a score on whether each condition is satisfied may be determined and integrated according to the weight, and when the integrated score exceeds a predetermined score, it may be determined if it is an orthostatic hypotension or not.

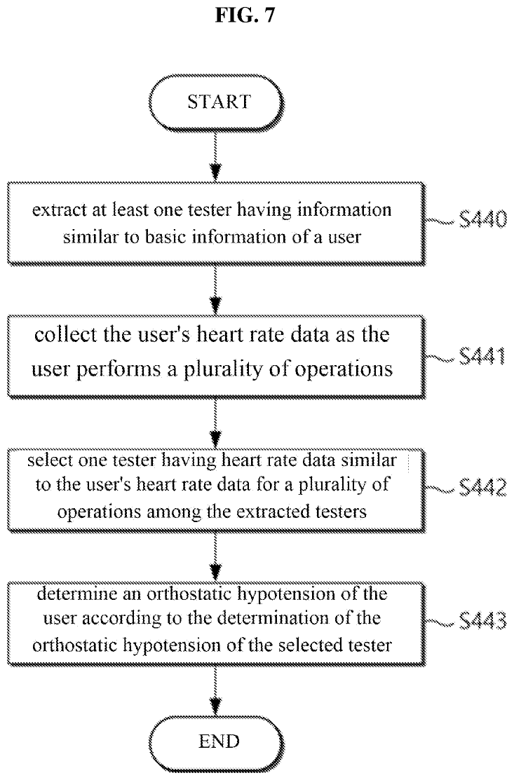

FIG. 7 is a diagram illustrating a method of determining orthostatic hypotension of a user according to another embodiment of the present invention.

Referring to FIG. 7, the computing device 100 may extract at least one tester having information similar to basic information of a user (S440). The computing device 100 may extract testers similar to the basic information of the user based on the basic information of the testers obtained from the testers. For example, the computing device 100 may calculate a similarity for each piece of basic information, and may extract testers in which a value obtained by combining the similarities is equal to or greater than a preset value. However, the present invention is not limited thereto, and a plurality of testers may be extracted in descending order of the similarity values, and testers classified into the same classification system as the user may be extracted.

The computing device 100 may collect the user's heart rate data as the user performs a plurality of operations (S441). A method of collecting the user's heart rate data may be performed in the same manner as described in step S200.

The computing device 100 may select one tester having heart rate data similar to the user's heart rate data for a plurality of operations among the extracted testers (S442). The computing device 100 may extract a tester having heart rate data most similar to the user's heart rate data based on the heart rate data of the testers obtained from the testers. For example, the computing device 100 may calculate the similarity of the heart rate data for each of the plurality of operations, and may extract one tester having the largest value obtained by integrating the similarities.

The computing device 100 may determine an orthostatic hypotension of the user according to the determination of the orthostatic hypotension of the selected tester (S443). For example, when it is determined that the selected tester is an orthostatic hypotension, the computing device 100 may determine that the user is also an orthostatic hypotension, and when it is determined that the selected tester is not an orthostatic hypotension, the computing device 100 may determine that the user is also not an orthostatic hypotension. Meanwhile, the computing device 100 may determine an orthostatic hypotension of the user according to the similarity value. For example, when the similarity between the basic information and the heart rate data between the selected tester and the user is low, when the orthostatic hypotension of the user is determined according to the judgment of the orthostatic hypotension of the selected tester, the judgment may be wrong. Accordingly, a probability of the orthostatic hypotension may be calculated based on the similarity, and the orthostatic hypotension of the user may be determined according to the calculated probability. In this case, the case in which the similarity is low may be determined according to a preset value, and the case in which the similarity is lower than the preset value may be determined to be low.

The present invention may accurately determine an orthostatic hypotension using heart rate data of a user measured by contact or non-contact.

More specifically, the present invention enables a user to determine orthostatic hypotension through a natural daily life without a separate event such as a Head up tilt test or a Valsalva maneuver, thereby increasing user convenience, and examining symptoms that occur in the daily life of a user.

The effects of the present invention are not limited to the effects mentioned above, and other effects not mentioned may be clearly understood by the skilled person in the art from the following description.

As described above, according to an embodiment of the present disclosure, a method, an apparatus, and a program for determining orthostatic hypotension using heart rate data for determining orthostatic hypotension using heart rate data measured by contact or non-contact may be realized.

Although the embodiments of the present invention have been described above with reference to the accompanying drawings, it will be understood that the skilled person in the art in the technical field to which the present invention pertains may be implemented in other specific forms without changing the technical idea or essential feature. Therefore, it should be understood that the above-described embodiments are exemplary and not restrictive in all aspects.

What is claimed is:

1. A method for determining orthostatic hypotension using heart rate data, wherein the method performed by a computing device comprises the steps of:
obtaining basic information of a user;
collecting heart rate data of the user;
obtaining activity information on an activity performed by the user in the process of collecting heart rate data; and
determining orthostatic hypotension of the user by analyzing the basic information, the heart rate data, and the activity information.

2. The method for determining orthostatic hypotension using heart rate data of claim 1, wherein the step of determining orthostatic hypotension comprises the steps of:
extracting input data from the basic information, heart rate data, and activity information;
inputting the input data to a pre-learned artificial intelligence model; and
obtaining an output of the artificial intelligence model and determining orthostatic hypotension of the user based on the output.

3. The method for determining orthostatic hypotension using heart rate data of claim 1, the method further comprising a step of collecting data for determining orthostatic hypotension for a plurality of testers, wherein the step of collecting the data comprises the steps of:
obtaining basic information of each testers;
collecting heart rate data according to operations performed by the testers and operations performed by the testers; and
determining orthostatic hypotension of each testers.

4. The method for determining orthostatic hypotension using heart rate data of claim 3, wherein the step of obtaining basic information of each testers comprises the steps of:
receiving physical information and disease information of the testers; and
classifying the testers according to a predetermined criterion based on the physical information and the disease information;
wherein the step of determining orthostatic hypotension of the testers comprises a step of calculating a possibility of occurrence of the orthostatic hypotension for each group classified according to a predetermined criterion based on the heart rate data.

5. The method for determining orthostatic hypotension using heart rate data of claim 4, wherein the step of determining orthostatic hypotension of the testers comprises the steps of:
extracting an operation in which the orthostatic hypotension occurs in each of the classification groups and a time during which the operation is performed; and
calculating an average of heart rate data collected when the operation is performed in each of the classification groups, and the step of determining orthostatic hypotension of the user comprises the steps of:
determining a classification group in which the user is included;
comparing heart rate data collected when the user performs an operation in which the orthostatic hypotension occurs in the classification group in which the user is included with the average of the heart rate data; and
determining orthostatic hypotension of the user according to a comparison result.

6. The method for determining orthostatic hypotension using heart rate data of claim 4, wherein the step of determining determining orthostatic hypotension of the testers comprises:
determining an operation in which the orthostatic hypotension has been generated by the testers included in each of the classification groups; and
setting a weight value with respect to the operation in which the orthostatic hypotension has been generated, wherein the step of determining the user's orthostatic hypotension comprises the steps of:
determining a classification group in which the user is classified based on basic information of the user;
assigning a score to heart rate data collected when the user performs a plurality of operations according to a score and a weight value corresponding to the classification group in which the user is included; and
determining that the user is the orthostatic hypotension when a score obtained by integrating scores of the plurality of operations exceeds a preset score.

7. The method for determining orthostatic hypotension using heart rate data of claim 3, wherein the step of collecting heart rate data comprises a step of collecting data obtained by sensing the heart rate data of the tester from a sensor attached to the body of the tester or an apparatus for measuring the heart rate data of the tester in a non-contact manner at a position spaced apart from the tester, wherein the step of determining orthostatic hypotension of the testers comprises the steps of:
providing questions about operations, respiration methods, and whether symptoms have occurred to each testers;
obtaining an answer to the questions from each testers; and
determining whether the orthostatic hypotension of each testers has occurred on the basis of the answer and extracting the heart rate data when the orthostatic hypotension has occurred.

8. The method for determining orthostatic hypotension using heart rate data of claim 7, wherein the step of extracting the heart rate data comprises the steps of:
determining a heart rate data pattern when the orthostatic hypotension occurs and before and after the occurrence of the orthostatic hypotension;
dividing the heart rate data pattern into a plurality of sections according to the heart rate data pattern; and
setting a range for determining orthostatic hypotension in each of the plurality of sections and a heart rate data difference range between the plurality of sections for determining orthostatic hypotension on the basis of basic information of the testers.

9. The method for determining orthostatic hypotension using heart rate data of claim 8, wherein the step of determining orthostatic hypotension of the user comprises the steps of:
determining a heart rate data area having a pattern similar to the heart rate data pattern from the collected heart rate data of the user;
dividing the heart rate data area into a plurality of sections according to the pattern of the heart rate data area;
extracting a difference between the heart rate data in each of the plurality of sections and the heart rate data between the plurality of sections; and
determining orthostatic hypotension of the user by determining whether the extracted data value is within the predetermined range.

10. The method for determining orthostatic hypotension using heart rate data of claim 3, wherein the step of determining orthostatic hypotension comprises the steps of:
extracting at least one tester having basic information similar to the basic information of the user;
collecting heart rate data of the user when the user performs a plurality of operations;
selecting one tester having heart rate data similar to the heart rate data of the user for the plurality of operations among the extracted testers; and
determining orthostatic hypotension of the user according to a determination of the orthostatic hypotension of the selected tester.

11. An apparatus comprising:
a memory configured to store one or more instructions; and
a processor configured to execute the one or more instructions stored in the memory, wherein the processor executes the one or more instructions to perform the method of claim 1.

12. A non-transitory computer readable storage medium storing instructions/program which, when executed by a hardware computer, cause the computer to carry out the method of claim 1.
